(19) United States
(12) Patent Application Publication
(10) Pub. No.: US 2008/0172105 A1
(43) Pub. Date: Jul. 17, 2008

(54) Method for preventing and/or ameliorating inflammation

(75) Inventors: Chih-Ching Lin, Taipei City (TW); Chyi-Ran Lee, Taipei City (TW)

Correspondence Address: Joe McKinney Muncy, PO Box 1364, Fairfax, VA, 22038-1364, US

(73) Assignee: WS FAR IR MEDICAL TECHNOLOGY CO., LTD.

(21) Appl. No.: 11/896,879

(22) Filed: Sep. 6, 2007

Related U.S. Application Data
(60) Provisional application No. 60/885,317, filed on Jan. 17, 2007.

(51) Int. Cl. A61B 18/18

(52) U.S. Cl. 607/50

(57) ABSTRACT

A method for preventing and/or ameliorating inflammation. The method comprises irradiating a biological subject with an electromagnetic wave from an emitter, wherein the electromagnetic wave has a wavelength of about 1.5 to 100 μm μm, and the biological subject can be a peripheral vascular disease patient. Additionally, the method of the invention can improve the access blood flow and unassisted patency of arteriovenous fistula in hemodialysis patients.

### BACKGROUND OF THE INVENTION

1. Field of the Invention

The present invention relates to the method for preventing inflammation, and in particular relates to a method for preventing and/or ameliorating inflammation by an electromagnetic wavelength commonly known as far-infrared.

2. Brief Description of the Related Art

Inflammation is the complex biological response of vascular tissue to harmful stimuli, such as pathogens, damaged cells, or irritants. It is a protective attempt by organisms to remove the harmful stimuli as well as initiate a healing process for the tissue. Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, immune system and various cells within the injured tissue. Prolonged inflammation, known as chronic inflammation, leads to a progressive shift in the type of cells which are present at the site of inflammation and is characterised by simultaneous destruction and healing of the tissue from the inflammatory process. Chronic inflammation can lead to many diseases and disorders, such as peripheral vascular disease.

Peripheral vascular disease commonly results from the build up of atherosclerotic plaque and/or thrombotic matter within peripheral arteries. In many cases, when arteries of the lower extremities have become obstructed by peripheral vascular disease, a phenomenon known as intermittent claudication results. Intermittent claudication is characterized by the occurrence of pain and progressive weakness in the legs during exertion (i.e., walking or running).

The typical surgical approach to the treatment of peripheral vascular disease, especially in patients who exhibit symptoms of intermittent claudication, is to surgically expose the affected artery and to anastomose a tubular bypass graft (e.g., a tube formed of woven polyester or expanded polytetrafluoroethylene (ePTFE)) to the affected artery such that one end of the graft is attached upstream of the obstruction, and the other end of the graft is attached downstream of the obstruction. In this manner, arterial blood will flow through the tubular bypass graft and around the arterial obstruction, thereby restoring blood flow to the portion of the artery downstream of the obstruction.

Infrared radiation is an invisible electromagnetic wave with a longer wavelength than that of visible light. According to the difference in wavelength, infrared radiation can be divided into three categories: near-infrared radiation (0.8-1.5 μm) and far-infrared (FIR) radiation (1.5-1000 μm). Infrared radiation transfers energy that is perceived as heat by thermoreceptors in the surrounding skin. However, no prior Art teaches or suggests a new use of FIR for the inhibition of inflammation and peripheral vascular disease.

### SUMMARY OF THE INVENTION

The invention provides a method for preventing and/or ameliorating inflammation, comprising irradiating a biological subject with electromagnetic wave from an emitter, wherein the electromagnetic wave has a wavelength of about 1.5 to 1000 μm, also known as far-infrared radiation.

The invention further provides a method for preventing and/or ameliorating peripheral vascular diseases caused by inflammation-induced vascular stenosis and/or thrombosis, comprising irradiating a skin of a biological subject with electromagnetic wave from an emitter, wherein the electromagnetic wave has a wavelength of about 1.5 to 1000 μm, also known as far-infrared radiation, and the biological subject is a peripheral vascular diseases patient.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the subsequent detailed description and the accompanying drawings, which are given by way of illustration only, and thus are not limitative of the present invention, and wherein:

FIGS. 1A-1B show that the far-infrared radiation induces Heme Oxygenase-1 (HO-1) expression;

FIGS. 2A-2B show that the far-infrared radiation induces Nrf2 expression;

FIG. 3 show that the far-infrared radiation enhances the promoter activity of a Heme Oxygenase-1 (HO-1) gene;

FIGS. 4A-4B show that the far-infrared radiation suppresses the expression of E-selectin, VCAM-1, and ICAM-1;

FIG. 5 show that-the far-infrared radiation suppresses TNF-α-induced VCAM-1;

FIG. 6A shows the relative endothelial adhesion of H-labeled U937 cells;

FIG. 6B shows the relative HO-1 expression in various groups, and

FIG. 7 shows one year survival curves for unassisted patency of AVF for HD patients with and without far-irradiation radiation treatment.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a method for preventing and/or ameliorating inflammation. The method comprises directly irradiating a biological subject with electromagnetic wave from an emitter, wherein the electromagnetic wave has a wavelength of about 1.5 to 1000 μm, also known as far-infrared (FIR) radiation. The inflammation can be an endothelial cell inflammation.

The far-infrared radiation (FIR) of the invention has a wavelength of about 1.5 to 100 μm, preferably about 3 to 25 μm, and a wavelength peak of about 5 to 8 μm, preferably about 5 to 6 μm. In the method of the invention, the distance between the emitter and the surface of the biological subject can be about 0.1 to 60 cm, preferably about 20 to 30 cm, wherein the surface can be a skin of an animal, such as a human. The power density of the emitter of the far-infrared radiation is lower than about 1.3 W/cm², preferably, about 0.5 to 0.7 W/cm². The irradiating time of far-infrared radiation each treatment session exceeds about 10 min, preferably, about 30 to 45 min. The treatment session frequency of far-infrared radiation exceeds once every two days, preferably about one to three times a day. The FIR of the invention can be obtained from any suitable emitter or radiator.

The method of the invention can prevent and/or ameliorate inflammation-related vascular disorders, including vascular diseases, peripheral vascular diseases, coronary artery disease, vasculitis including arteritis, phlebitis and thrombophlebitis, stenosis, atherosclerosis, thrombosis including venous thrombosis.

The “emitter or radiator” of the invention is a device used to emit electromagnetic wavelength also known as far-infrared radiation, and the shape and size of the emitter are not limited. The wavelengths of this electromagnetic wave are longer than microns and considered long wavelength radiation. Common emitting materials are ceramic oxides. They include magnesium oxide, aluminum silicates, silicon dioxide, iron oxide, aluminum oxide, zirconium oxide, and titanium dioxide. While these materials are good emitters, the radiation emitted is generally broad, covering typically 1.5 to 100 microns.

The term “inflammation” as used herein refers to all categories of inflammation, including localized manifestations and systemic inflammation; inflammation that is categorized temporally, e.g., chronic inflammation and acute inflammation; inflammation that is categorized in terms of its severity, e.g., mild, moderate, or severe; and inflammation that is a symptom or a result of a disease state or syndrome. Inflammation, as used herein, can be characterized at the “whole body” level as several localized manifestations, including hemodynamic disorders (e.g., hyperemia and edema), pain, temperature increment, and functional lesion. All manifestations may be observed in certain instances, although any particular manifestation may not always be present in all instances. Concomitant cellular and molecular level changes that characterize inflammation may include leukocyte extravasation and platelet aggregation. Molecular level changes which characterize in flammation may include activation of at least three plasma defense systems and synthesis of cytokines and eicosanoids.

The term “endothelium” of the invention refers to the layer of thin specialized epithelium, comprising a simple squamous layer of cells that line the interior surface of blood vessels, forming an interface between circulating blood in the lumen and the vessel walls. Endothelial calls line the entire circulatory system, from the heart to the smallest capillary. These cells reduce friction of the flow of blood allowing the fluid to be pumped further.

The “biological subject” of the invention refers to any living organism and any substance found within, purified from, or derived from any living organism, or any substance synthesized in vitro to recapitulate or resemble any substance found within, purified from, or derived from any living organism. The biological subjects include cell, tissue, organ, human or non-human mammal, e.g. a dog, a cat, a mouse, a rat, a cow, a sheep, a pig, a goat, or a primate, and expressly includes laboratory mammals, livestock, and domestic mammals. In some embodiments, the mammals may be a human; in others, the mammal may be a rodent, such as a mouse or a rat. The surface temperature of the biological object is about 30° C. to 45° C.

In one embodiment, the biological subject can be an inflammation-related vascular disorder patient. Inflammation-related vascular disorders include, but are not limited to, vascular diseases, peripheral vascular diseases, coronary artery disease, vasculitis including arteritis, phlebitis and thrombophlebitis, stenosis, atherosclerosis, thrombosis including venous thrombosis.

In another embodiment, the biological subject can be a peripheral vascular diseases patient. In medicine, peripheral vascular disease is a collator for all diseases caused by the obstruction of large peripheral arteries, which can result from atherosclerosis, inflammatory induced stenosis, an embolism or thrombus formation. The peripheral vascular diseases include, but are not limited to, peripheral artery occlusive diseases, diabetic arteriosclerosis obliterans, a arteriovenous fistula or graft dysfunction, varicosity, macroangiopathy and microangiopathy, cerebrovascular diseases, superficial thrombophlebitis, phlebitis, thromboangiitis obliterans (TAO or Buerger's disease), rheumatoid vasculitis, or Raynaud's syndrome.

In addition, FIR treatment of the invention has a lot of advantages, such as convenience, no operational risk, no observed side effects, and it is very safe and does not cause biological damage. A more effective use would be to combine FIR treatment of the invention with other treatment methods in order to improve therapeutic effectiveness. The so-called other treatment methods include administering steroidal anti-inflammatory drugs or Non-steroidal anti-inflammatory drugs (NSAIDs).

The method of the invention can also induce the expression of NrF2 and Heme Oxygenase-1 (HO-1) in human umbilical vein endothelial cells (HUVEC), with the expressions of NrF2 and HO-1 induced in a dose-dependent manner. After a 40 min. FIR treatment, the expression of NrF2 is about 6 to 8 times higher than the expression of NrF2 without FIR treatment, and the expression of HO-1 is increased about 2 to 4 times higher than the expression of HO-1 without FIR treatment. Additionally, the method of the invention suppresses the expression of E-selectin, VCAM-1 (vascular cell adhesion molecule-1), and ICAM-1 (intercellular adhesion molecule-1) in HUVEC. E-selectin, also known as CD62E, is a cell adhesion molecule expressed only on endothelial cells activated by cytokines. VCAM-1 also known as CD106, is a molecule with a considerable role in the human immune system. ICAM-1 is a type of intercellular adhesion molecule continuously present in low concentrations in the membranes of leukocytes and endothelial cells. After a 40 min. FIR treatment, the expression of E-selectin showed a decrease of about 7 to 9 times, the expression of VCAM-1 showed a decrease of about 20 to 25 times, and the expression of ICAM-1 showed a decrease of about 1 to 2 times. Thus, the method of the invention can suppress monocyte adhering to HUVECs, and therefore improve blood flow and patency.

Furthermore, the anti-inflammation effects of the method of the invention can improve blood flow and patency of arteriovenous fistula (AVF) in hemodialysis patients. Likewise, the method of the invention also improves the blood flow and patency of carotid artery and peripheral arteries in hemodialysis patients.

In another embodiment, the invention further provides a method for preventing and/or ameliorating peripheral vascular diseases caused by inflammation-induced vascular stenosis, comprising irradiating a skin of a biological subject with far-infrared radiation from an emitter to improve the blood flow and patency in the biological subject, wherein the far-infrared radiation has a wavelength of about 1.5 to 100 μm, and the biological subject is a peripheral vascular diseases patient.

The far-infrared radiation (FIR) has a wavelength of about 1.5 to 100 μm, preferably about 3 to 25 μm, and a wavelength peak of about 5 to 8 μm, preferably about 5 to 6 μm. The distance between the emitter and the skin can be about 0.1 to 60 cm, preferably about 20 to 30 μm. The power density of the far-infrared radiation is lower than about 1.3 W/cm², preferably, about 0.5 to 0.7 mW/cm². The irradiating time of far-infrared radiation exceeds about 10 min, preferably, about 30 to 45 min. The treatment session frequency of far-infrared radiation exceeds once every two days, preferably about one to three times a day. The inflammation can be an endothelial cell inflammation.

The term “vascular stenosis” of the invention refers to an abnormal narrowing in a blood vessel or other tubular organs or structures. Types of vascular stenosis are often associated with noise resulting from turbulent flow over the narrowed blood vessel. This bruit can be made audible by a stethoscope.

The peripheral vascular diseases of patient include, but are not limited to, peripheral artery occlusive diseases, diabetic arteriosclerosis obliterans, AV fistula dysfunctions, varicosity, macroangiopathy and microangiopathy, cerebrovascular diseases, superficial thrombophlebitis, phlebitis, thromboangiitis obliterans (TAO or Buerger's disease), rheumatoid vasculitis, or Raynaud's syndrome.

### EXAMPLE

### Example 1

### FIR Therapy Induces Heme Oxygenase-1 (HO-1) Expression in HUVECs

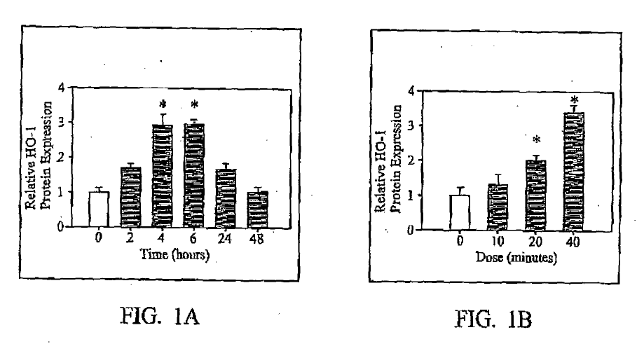

Human umbilical vein endothelial cells (HUVEC)(Walkersville, Md.) were serially cultured on gelatin-coated dishes and propagated in M199 medium supplemented with 20% bovine calf serum, 2 mM L-glutamine, 50 μg/ml endothelial cell growth factor, 90 μg/ml heparin, and 100 U/ml of penicillin and streptomycin. The cells were treated with FIR for 10 min, 20 min, 40 min using WS™ TY101 FIR emitter (WS Far Infrared Medical Technology Co., Ltd., Taipei, Taiwan). The top emitter was set at a height of 25 cm above the surface of the cells. After FIR treatment, the cells were collected at 0 h, 2 h, 4 h, 6 h, 24 h, and 48 h, and then the expression of HO-1 was analyzed by western blot and quantified by laser densitometry. The cells were lysed in a sample buffer (125 mM Tris [pH 6.8], 12.5% glycerol, 2% SDS, 50 mM sodium fluoride, and trace bromophenol blue) and proteins were separated by SDS-PAGE. Following transfer to a nitrocellulose membrane, blots were blocked with PBS and nonfat milk (5%) and then incubated with antibodies directed against HO-1 (1:500). Membranes were then washed in PBS, incubated with horseradish peroxidase-conjugated goat anti-rabbit or anti-goat antibody and developed with commercial chemoluminescence reagents (Amorsham, Arlington Heights, Ill.). FIG. 1A shows that the expression of HO-1 increased significantly at 4 h after 40 minutes of FIR treatment, with the maximum effect at 6 h and this was sustained until 24 h. FIG. 1B shows that 20 and 40 minutes of FIR treatment significantly induced higher expression of HO-1 compared to 0 and 10 minutes, with the maximum increase after 40 minutes of treatment. Thus, HO-1 expression was induced in a dose-dependent manner by FIR treatment.

### Example 2

### FIR Therapy Induces Nrf2 Expression in HUVECs

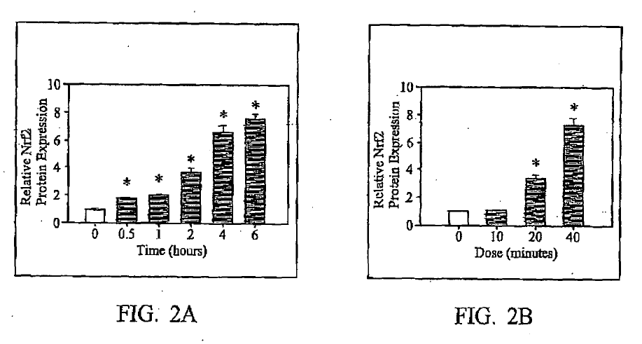

The same procedure carried out in Example 1 was repeated except that the expression of Nrf2 was detected. FIG. 2A shows that the increase of Nrf2 was time-dependent after 40 minutes of FIR therapy, with a significant increase from 0.5 h and a maximum effect at 6 h. FIG. 2B shows that 20 and 40 minutes of FIR treatment significantly induced higher expression of HO-1 than 0 and 10 minutes, with the maximum increase after 40 minutes of treatment. Thus, Nrf2 expression was induced in a dose-dependent manner by Flit treatment

### Example 3

### FIR Enhances Promoter Activity of HO-1 Gene Through Nrf2-Dependent Pathway

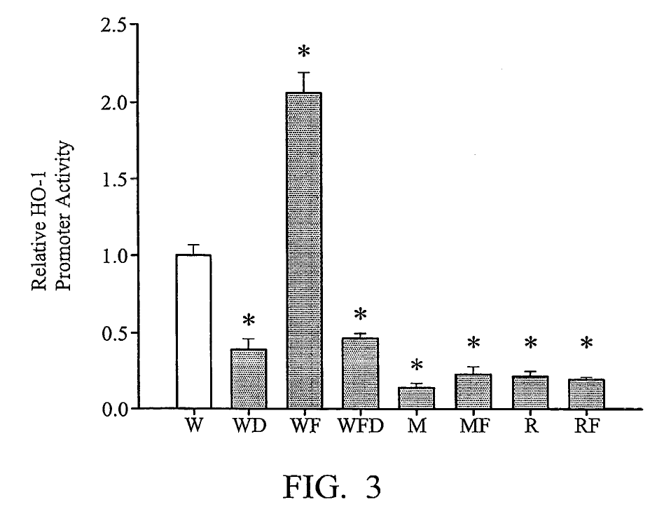

HO-1 promoter activity was determined in promoter/luciferase constructs (1 μg/ml) containing the wild type enhancer (E1) coupled to a minimum Heme Oxygenase-1 (HO-1) promoter or the mutant E1 enhancer (M739) that had its three antioxidant responsive element (ARE) core sequences mutated. These promoter constructs, pCMVβ-galactosidase (1 μg/ml), and a plasmid expressing a dominant-negative Nrf2 (dnNrf2; 1 μg/ml) that has its transactivation domain deleted were transfected into SMC using lipofectamine, and cells exposed to BRA 24 hours later. The cells were then collected, lysed, and luciferase activity measured using a dual luciferase assay system (Promega, Madison, Wis.) and a Glomax luminometer (Promega, Madison, Wis.). Firefly luciferase activity was normalized with respect to Renilla luciferase activity, and expressed as fold induction over control cells. All constructs were generously provided by Dr. Jawed Alan at the Ochsner Clinic Foundation, New Orleans, La. Referring to FIG. 3, In comparison with the wild plasmids (W) treated cells, FIR treated cells (W+F=WF) induced more than a 2-fold increase in promoter activity, which was significantly suppressed to less than 0.5-fold by adding DnNrf2 (W+F+D=WFD).

### Example 4

### FIR Treatment Suppresses TNF-α-Induced E-Selectin, VCAM-1 and ICAM-1 Expression in HUVECs

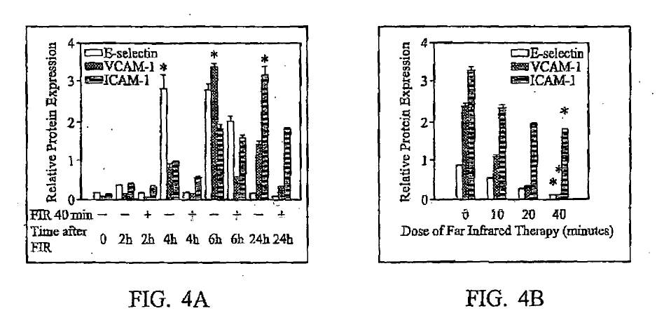

The same procedure carried out in Example 1 was repeated except that the expression of E-selectin, VCAM-1 and ICAM-1 were detected. FIG. 4A shows that the E-selectin, VCAM-1, and ICAM-1 were suppressed at hours 2, 4, 6, and 24 after 40 minutes of FIR treatment. FIG. 4B shows that the cells were collected at 4 hours for E-selectin, 6 hours for VCAM-1 and 24 hours for ICAM-1 after FIR treatment for 0, 10, 20 and 40 minutes, with the maximum inhibited response by 40 minutes of treatment for all of the three adhesion molecules. Thus, expression of E-selectin, VCAM-1 and ICAM-1 was suppressed in a dose-dependent manner by FIR treatment.

### Example 5

### FIR Suppresses TNF-α-Induced VCAM-1 Expression Through the HO-1 in HUVECs

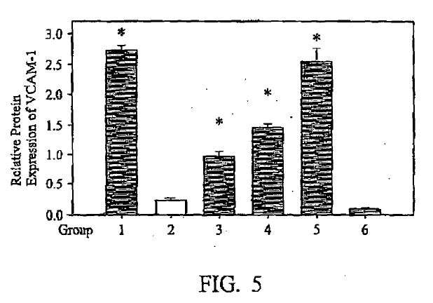

The expression of VCAM-1 was analyzed under different combinations of TNFα, FIR, HO-1 inhibitor by a western blot. HUVEC cells were classified into 6 groups according to the combination of different treatments, and the experimental condition of groups 1-6 are listed in Table 1. Referring to FIG. 5, the expression of VCAM-1 induced by TNF-α (100 ng/ml) in group 1 was significantly suppressed by adding 40 minutes of FIR in group 2. This FIR-induced inhibitory effect of VCAM-1 expression was reversed by tin protoporphyrin (SnPP, an HO-1 inhibitor) in a dose-dependent pattern, with a higher expression of VCAM-1 by 20 μM (group 4) than by 10 μM (group 3). In comparison with group 4, the expression of VCAM-1 was even higher without FIR therapy in group 5. However, a single treatment with SnPP 20 μM did not induce the expression of VCAM-1 without a pre-treatment with TNF-α (group 6).

TABLE 1

| | Group 1 | Group 2 | Group 3 | Group 4 | Group 5 | Group 6 |
|---|---|---|---|---|---|---|
| TNF-α | 100 | 100 | 100 | 100 | 100 | — |
| (ng/ml) | | | | | | |
| FIR (min) | — | 40 | 40 | 40 | — | — |
| SnPP (μm) | — | — | 10 | 20 | 20 | 20 |

### Example 6

### FIR Therapy Inhibits the Adhesion of Monocytes to HUVECs Through the Stimulation of HO-1 Expression

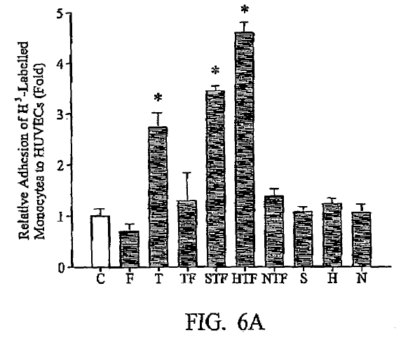

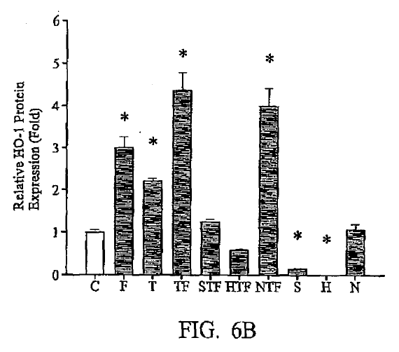

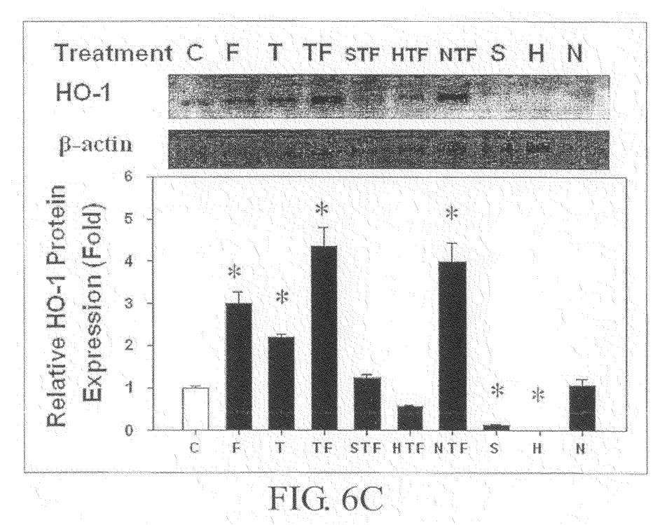

U937 cells were classified into 10 groups (F, T, TF, STF, HTF, NTF, S, H, N, or C) according to the combination of different treatment, and the experimental S conditions of groups are listed in Table 2. U937 cells (1×10⁶ cells/ml) were labeled with [³H]thymidine (1 μCi/ml) for 24 hour, and then washed 3 times with a serum-free culture medium and layered onto endothelial cell monolayers that were pretreated with TNFα (100 ng/ml) for 6 hours in the presence or absence of FIR and/or SnPP. After 1 hour of incubation, nonadherent monocytes were removed by PBS washing and the radioactivity associated with adherent cells were quantified by scintillation spectrometry after lysis with 0.2% SDS/0.2 N NaOH. Additionally, the expression of HO-1 was analyzed by western blot and quantified by laser densitometry. FIG. 6A shows the relative endothelial adhesion of H-labeled U937 cells, and FIG. 6B shows the relative HO-1 expression in various groups. Referring to FIG. 6B, the FIR treatment inhibits the adhesion of H-labeled U937 cells, and the adhesion of U937 cells were increased in groups T, STF, and HTF. Referring to 6C, the HO-1 expression of F group was approximately 3-fold higher than the control group, and TF group was 4.3-fold higher than the control group. However, the HO-1 expression of the cells treated by SnPP and HO-1 SiRNA were suppressed to approximately 20% and less than 10% of that of the control cells respectively.

TABLE 2

| | Group | | | | | | | | | |
|---|---|---|---|---|---|---|---|---|---|---|
| Treatment | F | T | TF | STF | HTF | NTF | S | H | N | C |
| TNF-α | − | 100 | 100 | 100 | 100 | 100 | − | − | − | − |
| (ng/ml) | | | | | | | | | | |
| FIR (min) | 40 | − | 40 | 40 | 40 | 40 | − | − | − | − |
| SnPP (μm) | − | − | − | 20 | − | + | 20 | − | − | − |
| Non-target | − | − | − | − | − | + | − | − | + | − |
| siRNA | | | | | | | | | | |

### Example 7

### Far-Infrared Radiation (FIR) Improves Access Blood flow and Unassisted Patency of Arteriovenous Fistula

Patient Selection

Patients included met the following criteria: (1) received 4 hours of maintenance HD therapy three times weekly for at least 6 months at Taipei Veterans General Hospital, (2) used a native AV fistula as their present vascular access for more than 6 months, without interventions within the last 3 months, and (3) creation of AV fistula by cardiovascular surgeons at Taipei Veterans General Hospital, was by standardized surgical procedures of venous end-to-arterial side anastomosis in the upper extremity. The study was based on the Helsinki Declaration [edition 6, revised 2000] and was approved by the Institutional Research Board of Taipei Veterans General Hospital. In addition, it was registered at the Cochrane Renal Group registry. After informed consent was obtained from every study subject, the patients were randomly allocated to either the group without FIR treatment or the group with FIR treatment by means of a computerized minimization algorithm to ensure balance between the two groups with respect to history of AV fistula malfunction. The subject allocation was concealed from investigators by the computerized minimization algorithm and the allocation sequence and was kept undisclosed to the investigator until the time of intervention.

Hemodialysis (HD)

All patients were dialyzed three times weekly on standard bicarbonate dialysate bath (38 mEq/L H₂CO₃, 3.0 mEq/L Ca²⁺, 2.0 mEq/L K⁺) by using the volumetric-controlled dialysis delivery system under constant dialysate flow at 500 ml/min. Patients were anticoagulated by means of systemic heparin, without change of the individual bolus or maintenance dose throughout the study.

Far-Infrared Radiation (FIR) Treatment.

A WS™ TY101 FIR emitter (WS Far Infrared Medical Technology Co., Ltd., Taipei, Taiwan) was used for FIR treatment. The wavelengths generated by the FIR emitter range from about 3 μm to about 25 μm (with a peak value of about 5 μm to about 7 μm). During treatment, the top of the FIR emitter was set at a height of about 25 cm above the skin surface of the AV fistula with the treatment time set at about 40 minutes during HD three times a week.

Measurement of Hemodynamic Parameters

The access flow (Qa), cardiac output (CO) and total peripheral resistance (TPR) were measured during HD by an ultrasound dilution method using the Transonic HD02 hemodialysis monitor (Transonic Systems, Inc., Ithaca, N.Y.). The technique is widely used and validated extensively in literature. In brief, the technique uses two ultrasound sensors attached to the two HD tubing lines, one to the arterial and the other to the venous catheters, approximately 3 to 5 inches from the connection of the tubing to the dialysis needles. Initially, tubing lines are reversed, and ultrafiltration is turned off. A measured bolus of saline (10 ml) is injected into the venous catheter, resulting in changes in sound velocity that are measured by the transducers on the catheters. The change is then calculated by the Transonic software, giving the result of Qa (ml/min). If Qa was unable to be obtained by the method, it was measured by the variable pump flow-based Doppler ultrasound method (Am J Kidney Dis 21:457-471, 1993). CO was measured by injecting 30 ml saline (37° C.) into the venous catheter without reversing the tubing lines. TPR was calculated by computer software by dividing the mean arterial blood pressure by CO.

Statistical Analyses

Data management and statistical analysis wore done using the SPSS statistical software (version 11.0; USA). Distributions of continuous variables in groups were expressed as mean±SD and compared by Student's t-test. All data have been tested for normal distribution before using t-tests. Categorical variables, such as the frequency of AV fistula malfunction in the treatment group and controls were analyzed by the chi-square test. The 95% confidence interval for every variable was also calculated. Survival curves of unassisted patency of AV fistula were calculated by the Kaplan-Meier method and compared by the log-rank test. A statistically significant value was P less than 0.05.

Patient Characteristics

145 patients were enrolled in the study. Among them, 73 patients were randomly distributed to the control group and 72 patients to the group treated by FIR. As listed in Table 3, there was no difference in the demographic and clinical characteristics between the two groups of HD patients. During follow-up, 1 patient receiving FIR therapy and 4 patients in the control group underwent creation of another vascular access because of the poor response to angioplasty. In addition, patients were censored at the time of renal transplantation (n=3), death with a functioning access (n=5), shifting to peritoneal dialysis (n=4), or loss of follow-up (n=1). This study was terminated on Dec. 31, 2005. Finally, 127 patients completed the study with 64 patients in the control group and 63 patients in the group treated with FIR therapy.

TABLE 3

| | Control | | P |
|---|---|---|---|
| | group | FIR group | value |
| N | 73 | 72 | |
| Age (years) | 59.2 ± 15.0 | 61.9 ± 14.4 | 0.87 |
| Gender (male) | 38 (52.1%) | 37 (51.4%) | 0.94 |
| HD duration (months) | 79.2 ± 42.2 | 85.2 ± 41.1 | 0.76 |
| Prevalence of hypertension | 39 (53.4%) | 40 (55.6%) | 0.80 |
| Prevalence of diabetes mellitus | 24 (32.9%) | 25 (34.7%) | 0.81 |
| History of AVF malfunctiona | 34 (46.6%) | 33 (45.8%) | 0.81 |
| Number of angioplasty | 20/46 | 20/49 | 0.87 |
| (patients/procedures) | | | |
| Number of surgical revision | 14/19 | 13/20 | 0.56 |
| (patients/procedures) | | | |
| Duration of AVF (months) | 58.7 ± 33.8 | 56.8 ± 36.4 | 0.91 |
| Creation of another AV fistula (n)^{b} | 4 (5.5%) | 1 (1.4%) | 0.18 |
| Renal transplantation (n) | 1 (1.4%) | 2 (2.8%) | 0.55 |
| Peritoneal dialysis (n) | 2 (2.7%) | 2 (2.8%) | 0.99 |
| Death with functioning AV fistula (n) | 2 (2.7%) | 3 (4.2%) | 0.64 |
| Loss to follow-up (n) | 0 | 1 (1.4%) | 0.31 |

^{b}Creation of another AV fistula during this 1-year study

The Effect of Single Session of FIR Therapy on the Hemodynamic Parameters in HD Patients

There was no significant difference in most of the hemodynamic parameters (such as blood pressure, cardiac output, total peripheral resistance) between the single HD session with or without FIR treatment for the 72 HD patients. However, the incremental change of access flow [Δ(Qa2-Qa1)] of these patients in the single HD session with FIR therapy was significantly higher than that without FIR therapy (13.2±114.7 vs. −33.4±132.3 ml/min; P=0.021), the results are listed in Table 4.

TABLE 4

| | HD session | HD session | |
|---|---|---|---|
| | without FIR | with FIR | P value |
| SBP1 (mmHg) | 134.2 ± 23.9 | 130.7 ± 19.0 | 0.86 |
| SBP2 (mmHg) | 132.1 ± 19.1 | 129.4 ± 17.3 | 0.74 |
| Δ(SBP2 − SBP1) (mmHg) | −2.1 ± 14.5 | −1.3 ± 14.8 | 0.68 |
| Qa1 (ml/min) | 976.6 ± 491.2 | 967.8 ± 421.0 | 0.94 |
| Qa2 (ml/min) | 943.2 ± 472.2 | 981.0 ± 430.8 | 0.36 |
| Δ(Qa2 − Qa1) (ml/min) | −33.4 ± 132.3 | 13.2 ± 114.7 | 0.021 |
| CO1 (L/min) | 4.75 ± 1.38 | 4.72 ± 1.10 | 0.91 |
| CO2 (L/min) | 4.45 ± 1.25 | 4.34 ± 1.16 | 0.88 |
| Δ(CO2 − CO1) (L/min) | −0.30 ± 0.75 | −0.38 ± 0.74 | 0.77 |
| Qa1/CO1 | 0.223 ± 0.090 | 0.217 ± 0.076 | 0.78 |
| Qa2/CO2 | 0.216 ± 0.082 | 0.235 ± 0.099 | 0.42 |
| Δ[(Qa2/CO2) − | −0.007 ± 0.057 | 0.018 ± 0.056 | 0.027 |
| (Qa1/CO1)] | | | |
| TPR1 (mmHg × min/L) | 21.29 ± 5.56 | 20.86 ± 4.81 | 0.89 |
| TPR2 (mmHg × min/L) | 22.86 ± 6.09 | 22.67 ± 5.57 | 0.92 |
| Δ(TPR2 − TPR1) | 1.56 ± 4.35 | 1.82 ± 3.98 | 0.81 |
| (mmHg × min/L) | | | |

SBP: systolic blood pressure;
Qa: access flow;
CO: cardiac output;
TPR: total peripheral resistance;
1 indicates the timing of measuring the parameter is within the first hour after initiation of HD session or immediately before FIR treatment;
2 represents the timing of measuring the parameter is 40 minutes after timing 1 or immediately after FIR treatment;
NS: not significant

The Effect of One Year of FIR Therapy on Access Flow And Unassisted Patency of AV Fistula in HD Patients

In comparison with the control group, the HD patients receiving FIR treatment thrice a weak for a year had higher incremental values of the following changes of access flow regarding both the initial and final HD sessions, including (1) Δ(Qa4-Qa3) [36.2±82.4 vs. −12.7±153.6 ml/min; P=0.027], (2) Δ(Qa3-Qa1) [36.3±166.2 vs. −51.7±283.1 ml/min; P=0.035], (3) Δ(Qa4-Qa2) [99.2±144.4 vs. −47.5±244.5 ml/min; P<0.001], and (4) Δ(Qa4-Qa2)−Δ(Qa3-Qa1) (ml/min) [62.9±111.6 vs. 4.1±184.5 ml/min; P=0.032], the results are listed in Table 5. As listed in Table 4, the FIR group had a lower incidence of AV fistula malfunction [12.5% ( 9/72) vs. 30.1% ( 22/73); P<0.01]. Some patients experienced multiple episodes of AV fistula malfunction during this study. Thus, relative incidences of AV fistula malfunction (number of incidences per patient months of follow-up) were calculated. The relative incidence of AV fistula malfunction in the FIR group (1 episode per 67.7 patient-months) was significantly lower than that in the control group (1 episode per 26.7 patient-months; P=0.03; Table 4).

TABLE 5

| | Control group | FIR group | P value |
|---|---|---|---|
| Case number completing study | 64 | 63 | — |
| Qa1 (ml/min) | 992.8 ± 473.8 | 975.2 ± 421.9 | 0.58 |
| Qa2 (ml/min) | 975.9 ± 444.3 | 948.6 ± 432.7 | 0.26 |
| Δ(Qa2 − Qa1) (ml/min) | −16.9 ± 130.3 | −26.6 ± 105.5 | 0.72 |
| Qa1/CO1 | 0.206 ± 0.088 | 0.204 ± 0.078 | 0.90 |
| Qa2/CO2 | 0.223 ± 0.095 | 0.225 ± 0.107 | 0.93 |
| Δ[(Qa2/CO2) − (Qa1/CO1)] | 0.017 ± 0.060 | 0.021 ± 0.059 | 0.75 |
| Qa3 (ml/min) | 941.1 ± 367.9 | 1011.6 ± 447.0 | 0.14 |
| Qa4 (ml/min) | 928.4 ± 387.5 | 1047.8 ± 463.2 | 0.07 |
| Qa3/CO3 | 0.194 ± 0.071 | 0.213 ± 0.077 | 0.16 |
| Qa4/CO4 | 0.197 ± 0.074 | 0.237 ± 0.079 | 0.004 |
| Δ(Qa4 − Qa3) (ml/min) | −12.7 ± 153.6 | 36.2 ± 82.4 | 0.027 |
| Δ[(Qa4/CO4) − (Qa3/CO3)] | 0.003 ± 0.047 | 0.024 ± 0.033 | 0.004 |
| Δ(Qa3 − Qa1) (ml/min) | −51.7 ± 283.1 | 36.3 ± 166.2 | 0.035 |
| Δ[(Qa3/CO3) − (Qa1/CO1)] | −0.012 ± 0.064 | 0.009 ± 0.040 | 0.031 |
| Δ(Qa4 − Qa2) (ml/min) | −47.5 ± 244.5 | 99.2 ± 144.4 | <0.001 |
| Δ[(Qa4/CO4) − (Qa2/CO2)] | −0.026 ± 0.056 | 0.013 ± 0.060 | <0.001 |
| Δ(Qa4 − Qa2) − Δ(Qa3 − Qa1) (ml/min) | 4.1 ± 184.5 | 62.9 ± 111.6 | 0.032 |
| Δ[(Qa4/CO4) − (Qa2/CO2)] − | −0.014 ± 0.068 | 0.004 ± 0.068 | 0.049 |
| Δ[(Qa3/CO3) − (Qa1/CO1)] | | | |
| Case number starting study | 73 | 72 | — |
| Patients with new AVF malfunction | 22 (30.1%) | 9 (12.5%) | <0.01 |
| (%) | | | |
| Patients with thrombosis of AVF | 6 (8.2%) | 2 (2.8%) | 0.15 |
| Patients with intervention of AVF | 16 (21.9%) | 7 (9.7%) | 0.044 |
| Total observations (patient-months) | 802.4 | 812.6 | — |
| New episodes of AVF malfunction | 30 | 12 | — |
| during study | | | |
| Episode of AVF malfunction/patient- | 1/26.7 | 1/67.7 | 0.03 |
| months | | | |

Qa1 indicates the access flow measured within first hour after the initiation of the HD session immediately before the commencement of this study.
Qa2 indicates the access flow measured 40 minutes after Qa1 measurement during the HD session immediately before the commencement of this study.
Qa3 indicates the access flow measured before FIR treatment or within first hour after the initiation of the HD session when the study was completed.
Qa4 indicates the access flow measured 40 minutes after Qa3 or immediately after FIR treatment during the HD session when the study was completed.
Δ(Qa4 − Qa3) represents the sum of the thermal effect by FIR and the hemodynamic effect by HD on the change of Qa.
Δ(Qa3 − Qa1) represents the non-thermal effect of one year of FIR on the change of Qa.
Δ(Qa4 − Qa2) represents the sum of the thermal effect (for 40 minutes) and the non-thermal effect (for one year) by FIR on Qa.
[Δ(Qa4 − Qa2) − Δ(Qa3 − Qa1)] represents the thermal effect of 40 minutes of FIR on the change of Qa.

Access Survival

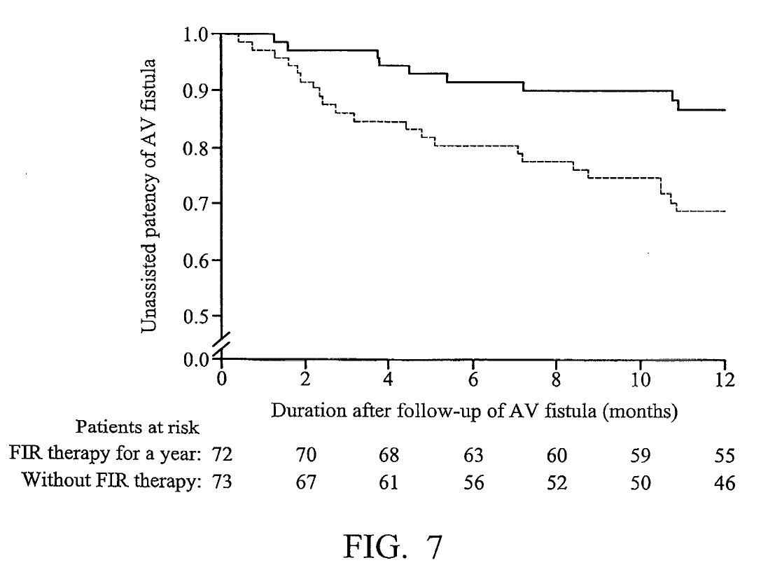

Eight patients of the FIR group and five patients of the control group were censored because of reasons other than AV fistula malfunction, such as renal transplantation, death with a functioning access, switching to peritoneal dialysis, and loss to follow-up. Consequently, the expected patient numbers used as the denominator for calculation of the unassisted patency of AV fistula was adjusted to 64 for the FIR group and 63 for the control group. Referring to FIG. 7, the unassisted patency of AV fistula at one year was significantly better in the FIR group than in the control group [85.9% (55 of 64) versus 67.6% (46 of 68); P<0.01 by Log rank test]. Concerning the safety issue of FIR therapy, no patient complained of any side effect, such as skin burn or allergy to the FIR therapy throughout the entire course of the study.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for preventing and/or ameliorating inflammation, comprising irradiating a biological subject with an electromagnetic wave from an emitter to inhibit inflammation in the biological subject, wherein the electromagnetic wave has a wavelength range of about 1.5 to 100 μm.

2. The method as claimed in claim 1, wherein the distance between the emitter and the surface of the biological subject is about 0.1 to 60 cm.

3. The method as claimed in claim 1, wherein the electromagnetic wave has a peak in a wavelength of about 5 to 8 μm.

4. The method as claimed in claim 1, wherein the surface temperature of the biological object is about 30° C. to 45° C.

5. The method as claimed in claim 1, wherein the power density of the emitter of the electromagnetic wave is lower than 1.3 W/cm².

6. The method as claimed in claim 1, wherein the irradiating time of the electromagnetic wave exceeds about 10 minutes per treatment session.

7. The method as claimed in claim 1, wherein the treatment frequency of the electromagnetic wave is at least once every two days.

8. The method as claimed in claim 1, wherein the inflammation is endothelial cell inflammation.

9. The method as claimed in claim 1, wherein the biological subject comprises a mammalian.

10. The method as claimed in claim 1, wherein the biological subject is a human.

11. The method as claimed in claim 1, wherein the biological subject is an inflammation-related cardiovascular disorders patient and/or peripheral vascular diseases patient.

12. The method as claimed in claim 11, wherein the inflammation-related cardiovascular disorders comprises vascular diseases, peripheral vascular diseases, coronary artery disease, vasculitis including arteritis, phlebitis and thrombophlebitis, stenosis, atherosclerosis, or thrombosis including venous thrombosis.

13. The method as claimed in claim 12, wherein the peripheral vascular diseases comprises peripheral artery occlusive diseases, diabetic arteriosclerosis obliterans, a arteriovenous fistula or graft dysfunction, varicosity, macroangiopathy and microangiopathy, cerebrovascular diseases, superficial thrombophlebitis, phlebitis, thromboangiitis obliterans (TAO or Buerger's disease), rheumatoid vasculitis, or Raynaud's syndrome.

14. A method for preventing and/or ameliorating peripheral vascular diseases caused by an inflammation-induced vascular stenosis, comprising irradiating a skin of a biological subject with an electromagnetic wave from an emitter to improve blood flow and patency in the biological subject, wherein the electromagnetic wave has a wavelength of about 1.5 to 100 μm, and the biological subject is a peripheral vascular disease patient.

15. The method as claimed in claim 14, wherein the distance between the emitter and the surface of the biological subject is about 0.1 to 60 cm.

16. The method as claimed in claim 14, wherein the electromagnetic wave has a peak in a wavelength of about 5 to 8 μm.

17. The method as claimed in claim 14, wherein the surface temperature of the biological object is about 30° C. to 45° C.

18. The method as claimed in claim 14, wherein the power density of the emitter of the electromagnetic wave is lower than 1.3 W/cm².

19. The method as claimed in claim 14, wherein the irradiating time of the electromagnetic wave exceeds 10 minutes per treatment session.

20. The method as claimed in claim 14, wherein treatment frequency of the electromagnetic wave radiation is at least once every two days.

21. The method as claimed in claim 14, wherein the inflammation is an endothelial cell inflammation.

22. The method as claimed in claim 14, wherein the peripheral vascular diseases comprises peripheral artery occlusive diseases, diabetic arteriosclerosis obliterans, a arteriovenous fistula or graft dysfunction, varicosity, macroangiopathy and microangiopathy, cerebrovascular diseases, superficial thrombophlebitis, phlebitis, thromboangiitis obliterans (TAO or Buerger's disease), rheumatoid vasculitis, or Raynaud's syndrome.
